(19) United States
(12) Patent Application Publication
(10) Pub. No.: US 2025/0146039 A1
(43) Pub. Date: May 8, 2025

(54) Terpene Glycoside Derivatives and Uses Thereof

(71) Applicant: Firmenich SA, Satigny (CH)

(72) Inventors: Dan-Ting YIN, Shanghai (CN); Xian-Wen GAN, Shanghai (CN)

(73) Assignee: Firmenich SA, Satigny (CH)

(21) Appl. No.: 19/020,457

(22) Filed: Jan. 14, 2025

Related U.S. Application Data
(63) Continuation of application No. 17/045,141, filed on Oct. 2, 2020.

(30) Foreign Application Priority Data
Aug. 22, 2018 (WO) PCT/CN2018/101661

(51) Int. Cl. C12P 19/44; A23L 27/30; C12P 19/18

(52) U.S. Cl. CPC C12P 19/44; A23L 27/36; C12P 19/18

(57) ABSTRACT

The present disclosure relates generally to terpene glycosides, such as certain such compounds extracted from Stevia rebaudiana Bertoni, Rubus suavissimus, or Siraitia grosvenorii. The disclosure also provides for the use of such compounds as food ingredients, flavors, and sweeteners, and related methods. The disclosure also provides ingestible compositions comprising such compounds, as well as processes for extracting such compounds selectively from certain plant sources.

### CROSS-REFERENCE TO RELATED APPLICATIONS

The present application is a continuation of U.S. patent application Ser. No. 17/045,141, filed Oct. 2, 2020, which is the U.S. national stage application of PCT Application No. PCT/EP2019/072356, filed Aug. 21, 2019, which claims the benefit of priority of PCT Application No. PCT/CN2018/101661, filed Aug. 22, 2018, each of which is hereby incorporated by reference as though set forth herein in their entirety.

### TECHNICAL FIELD

The present disclosure relates generally to terpene glycosides, such as certain such compounds extracted from Stevia rebaudiana Bertoni, Rubus suavissimus, or Siraitia grosvenorii. The disclosure also provides for the use of such compounds as food ingredients, flavors, and sweeteners, and related methods. The disclosure also provides ingestible compositions comprising such compounds, as well as processes for extracting such compounds selectively from certain plant sources.

### DESCRIPTION OF RELATED ART

The taste system provides sensory information about the chemical composition of the external world. Taste transduction is one of the more sophisticated forms of chemically triggered sensation in animals. Signaling of taste is found throughout the animal kingdom, from simple metazoans to the most complex of vertebrates. Mammals are believed to have five basic taste modalities: sweet, bitter, sour, salty, and umami.

Sweetness is the taste most commonly perceived when eating foods rich in sugars. Mammals generally perceive sweetness to be a pleasurable sensation, except in excess. Caloric sweeteners, such as sucrose and fructose, are the prototypical examples of sweet substances. Although a variety of no-calorie and low-calorie substitutes exist, these caloric sweeteners are still the predominant means by which comestible products induce the perception of sweetness upon consumption.

Metabolic disorders and related conditions, such as obesity, diabetes, and cardiovascular disease, are major public health concerns throughout the world. And their prevalence is increasing at alarming rates in almost every developed country. Caloric sweeteners are a key contributor to this trend, as they are included in various packaged food and beverage products to make them more palatable to consumers. In many cases, no-calorie or low-calorie substitutes can be used in foods and beverages in place of sucrose or fructose. Even so, these compounds impart sweetness differently from caloric sweeteners, and a number of consumers fail to view them as suitable alternatives. Moreover, such compounds may be difficult to incorporate into certain products. In some instances, they may be used as partial replacements for caloric sweeteners, but their mere presence can cause many consumers to perceive an unpleasant astringency. Therefore, lower-calorie sweeteners still face certain challenges to their adoption.

Terpene glycosides, such as steviol glycosides from Stevia (Stevia rebaudiana Bertoni) extracts, rubusoside from blackberry leaf (Rubus suavissimus) extracts, and mogrosides from monk fruit (Siraitis grosvenorii) extracts, are natural low-calorie sweeteners. But these products, like many other low-calorie sugar alternatives, have negative taste attributes, such as bitterness, lingering aftertaste, or licorice flavor. Transglucosylation provides a way of mitigating some of these negative taste attributes. But many of the presently disclosed glucosylated low-calorie sweeteners continue to exhibit negative taste attributes that prevent their widespread adoption. Thus, there is a continuing need to develop glucosylated products and transglucosylation methods that can provide more effective mitigation of negative taste attributes.

### SUMMARY

The present disclosure provides transglucosylation methods and glucosylated natural low-calorie sweeteners having an improved taste profile relative to other glucosylated derivatives of such compounds.

In a first aspect, the disclosure provides methods of making a glucosylated terpene glycoside, the method comprising: (a) providing an aqueous composition comprising an α-glucosyl sugar compound, a terpene glycoside, and a transglucosidase enzyme; and (b) reacting the α-glucosyl sugar compound with the terpene glycoside in the presence of the transglucosidase enzyme to form a glucosylated terpene glycoside having a terpene glycosidyl moiety and one or more α-glucosyl sugar moieties, wherein the glucosylated terpene glycoside has one α-1,6 glucosidic bond between the terpene glycoside moiety and one of the one or more α-glucosyl sugar moieties. In some embodiments, the reacting comprises incubating the aqueous composition. In some embodiments, the aqueous composition is an aqueous solution.

In a second aspect, the disclosure provides methods of reducing an unpleasant taste of a terpene glycoside, the method comprising: (a) providing an aqueous composition comprising an α-glucosyl sugar compound, a terpene glycoside, and a transglucosidase enzyme; and (b) reacting the α-glucosyl sugar compound with the terpene glycoside in the presence of the transglucosidase enzyme to form a glucosylated terpene glycoside having a terpene glycosidyl moiety and one or more α-glucosyl sugar moieties, wherein the glucosylated terpene glycoside has one α-1,6 glucosidic bond between the terpene glycoside moiety and one of the one or more α-glucosyl sugar moieties. In some embodiments, the reacting comprises incubating the aqueous composition. In some embodiments, the aqueous composition is an aqueous solution.

In some embodiments of the first or second aspects, the terpene glycoside is selected from the group consisting of: stevioside, rebaudioside A, rebaudioside B, rebaudioside C, rebaudioside D, rebaudioside E, rebaudioside F, rebaudioside G, rebaudioside M, dulcoside A, steviolbioside, rubusoside, terpene glycosides of Stevia rebaudiana Bertoni plants, terpene glycosides of Rubus suavissimus plants, terpene glycosides of Siraitis grosvenorii plants, and any combinations thereof.

In some embodiments of the first or second aspects, the alpha-glucosyl sugar compound is selected from the group consisting of: maltose, maltotriose, maltotetraose, partial hyrdrolysates of statch, maltodextrin, glucose, and sucrose.

In some embodiments of the first or second aspects, the transglucosidase enzyme is Aspergillus niger glucosyltransferase.

In some embodiments of the first or second aspects, the glucosylated terpene glycoside is selected from the group consisting of: mono α-1,6 glucosylated stevioside, mono α-1,6 glucosylated rebaudioside A, mono α-1,6 glucosylated rebaudioside B, mono α-1,6 glucosylated rebaudioside C, mono α-1,6 glucosylated rebaudioside D, mono α-1,6 glucosylated rebaudioside E, mono α-1,6 glucosylated rebaudioside F, mono α-1,6 glucosylated rebaudioside G, mono α-1,6 glucosylated rebaudioside M, mono α-1,6 glucosylated dulcoside A, mono α-1,6 glucosylated steviolbioside, mono α-1,6 glucosylated rubusoside, and combinations thereof.

In a third aspect, the disclosure provides a compound of formula I:

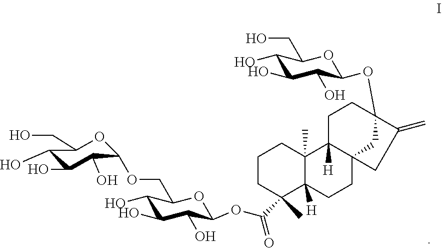

In a fourth aspect, the disclosure provides a compound of formula II:

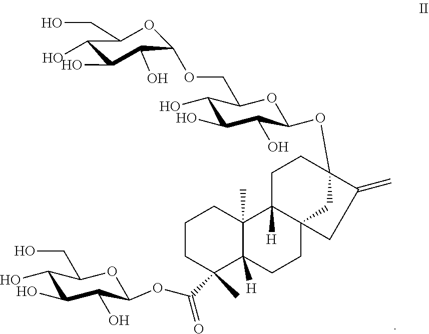

In a fifth aspect, the disclosure provides a compound of formula III:

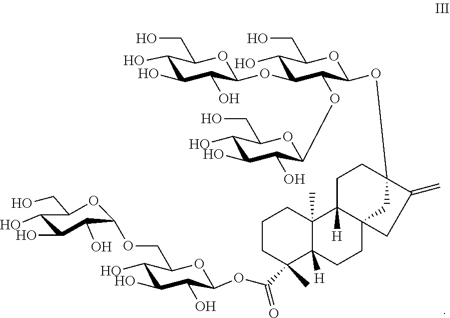

In a sixth aspect, the disclosure provides a compound of formula IV:

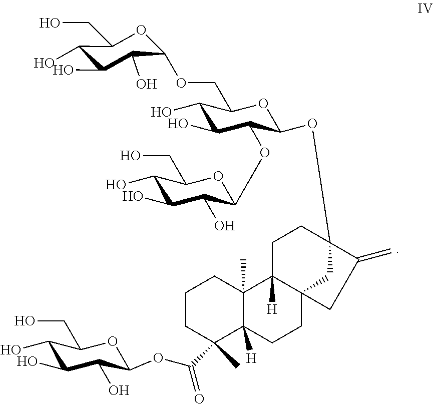

In a seventh aspect, the disclosure provides a compound of formula V:

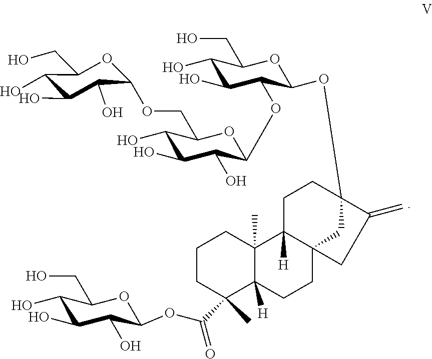

In an eighth aspect, the disclosure provides a compound of formula VI:

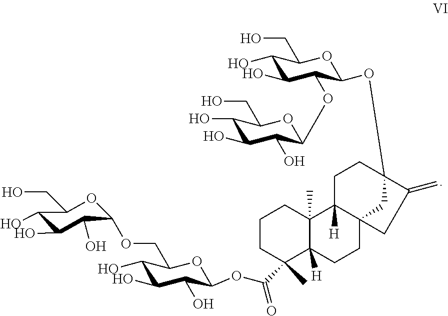

In a ninth aspect, the disclosure provides a compound of formula VII:

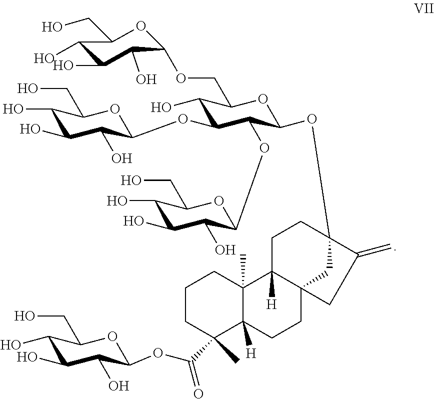

In a tenth aspect, the disclosure provides a compound of formula:

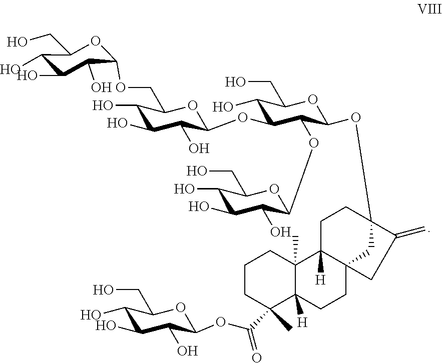

In an eleventh aspect, the disclosure provides a compound of formula IX:

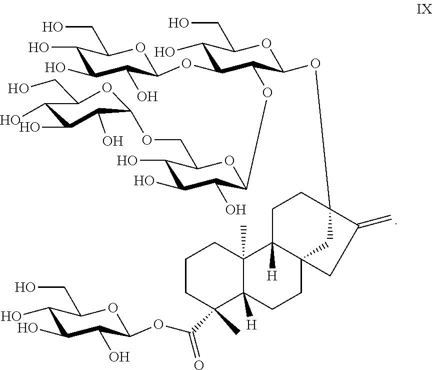

In a twelfth aspect, the disclosure provides a composition comprising at least one glucosylated terpene glycoside having a single α-1,6 glucosidic bond is selected from the group consisting of: mono α-1,6 glucosylated stevioside, mono α-1,6 glucosylated rebaudioside A, mono α-1,6 glucosylated rebaudioside B, mono α-1,6 glucosylated rebaudioside C, mono α-1,6 glucosylated rebaudioside D, mono α-1,6 glucosylated rebaudioside E, mono α-1,6 glucosylated rebaudioside F, mono α-1,6 glucosylated rebaudioside G, mono α-1,6 glucosylated rebaudioside M, mono α-1,6 glucosylated dulcoside A, mono α-1,6 glucosylated steviolbioside, and mono α-1,6 glucosylated rubusoside. In some further embodiments thereof, the composition comprises one or more compounds of any of the third through the eleventh aspects. In some other embodiments, the composition comprises one or more compounds made by a method of the first or second aspect. In some further embodiments, the composition comprises a compound of formula I, a compound of formula II, a compound of formula III, or a compound of formula VIII. In some further embodiments of any of the foregoing embodiments, the glucosylated terpene glycosides in the composition confer, enhance, improve, or modify a sweet taste of a flavored article. In some such embodiments, the terpene glycosides are present in the composition in an amount effective to confer, enhance, improve, or modify the sweet taste of the composition. In some embodiments, the composition is a flavored article. In some embodiments, the composition is not a naturally occurring composition.

In a thirteenth aspect, the disclosure provides uses of any of the compounds of the third through the eleventh aspects, or any compounds made according to the first or second aspects, to enhance the sweetness of a composition, such as an ingestible composition. In some embodiments thereof, the composition comprises a sweetener, such as a non-caloric or caloric sweetener.

Further aspects and embodiments of the present disclosure are set forth in the Detailed Description, the Drawings, and the Claims.

### BRIEF DESCRIPTION OF THE DRAWINGS

The following drawings are provided for purposes of illustrating various embodiments of the compositions and methods disclosed herein. The drawings are provided for illustrative purposes only, and are not intended to describe any preferred compositions or preferred methods, or to serve as a source of any limitations on the scope of the claimed inventions.

FIG. 1 shows an HPLC chromatogram of the product enzymatically generated by transglucosidase with Rubusoside (Rubu) and corn maltodextrin having a dextrose equivalent (DE) of 18 as substrates.

FIG. 2 shows an HPLC chromatogram of the product enzymatically generated by transglucosidase with Rubusoside (Rubu) and maltose as substrates.

FIG. 3 shows an HPLC chromatogram of the product enzymatically generated by transglucosidase with Rebaudioside A (RebA) and corn maltodextrin having a dextrose equivalent (DE) of 18 as substrates.

FIG. 4 shows an HPLC chromatogram of the product enzymatically generated by transglucosidase with a 90% pure composition of steviol glycosides (Layn, 60% of RebA) and corn maltodextrin having a dextrose equivalent (DE) of 18 as substrates.

FIG. 5 shows the ¹H NMR spectra for a mixture of the compound of formula I and the compound of formula II.

FIG. 6 shows the ¹³C NMR spectra for a mixture of the compound of formula I and the compound of formula II.

FIG. 7 shows the ¹H NMR spectra for the compound of formula III.

FIG. 8 shows the ¹³C NMR spectra for the compound of formula III.

FIG. 9 shows the ¹H NMR spectra for the compound of formula VIII.

FIG. 10 shows the ¹³C NMR spectra for the compound of formula VIII.

### DETAILED DESCRIPTION

In the following description, reference is made to specific embodiments which may be practiced, which is shown by way of illustration. These embodiments are described in detail to enable those skilled in the art to practice the invention described herein, and it is to be understood that other embodiments may be utilized and that logical changes may be made without departing from the scope of the aspects presented herein. The following description of example embodiments is, therefore, not to be taken in a limited sense, and the scope of the various aspects presented herein is defined by the appended claims. The Abstract is provided to allow the reader to quickly ascertain the nature and gist of the technical disclosure. The Abstract is submitted with the understanding that it will not be used to interpret or limit the scope or meaning of the claims.

### Methods

In certain aspects, the disclosure provides methods of making a glucosylated terpene glycoside, the method comprising: (a) providing an aqueous composition comprising an α-glucosyl sugar compound, a terpene glycoside, and a transglucosidase enzyme; and (b) reacting the α-glucosyl sugar compound with the terpene glycoside in the presence of the transglucosidase enzyme to form a glucosylated terpene glycoside having a terpene glycosidyl moiety and one or more α-glucosyl sugar moieties, wherein the glucosylated terpene glycoside has one α-1,6 glucosidic bond between the terpene glycoside moiety and one of the one or more α-glucosyl sugar moieties. In some such aspects, the methods are methods of reducing an unpleasant taste of a terpene glycoside.

In some embodiments thereof, the disclosure provides enzymatic processes for making an composition comprising glucosylated forms of stevioside, rebaudioside A, rebaudioside B, rebaudioside C, rebaudioside D, rebaudioside E, rebaudioside F, rebaudioside M, dulcoside A, steviolbioside, rubusoside, terpene glycosides of Stevia rebaudiana Bertoni plants, terpene glycosides of Rubus suavissimus plants, terpene glycosides of Siraitis grosvenorii plants, or any combinations thereof.

In some embodiments, a starting material for the enzymatic process is an extract of a Stevia rebaudiana Bertoni plant, or an extract of a Rubus suavissimus plant, or an extract of a Siraitis grosvenorii plant. In some further embodiments, the plant extracts contain one or more terpene glycosides.

For example, by way of non-limiting illustration, Stevia rebaudiana Bertoni, produces a number of diterpene glycosides, including stevioside, rebaudioside A, rebaudioside B, rebaudioside C, rebaudioside D, rebaudioside E, rebaudioside F, rebaudioside G, rebaudioside M, dulcoside A, and steviolbioside. As another non-limiting example, rubusoside may be obtained from blackberry leaves (Rubus suavissimus), which substantially contain a single terpene glycoside called rubusoside. In some cases, rubusoside is also present in low amounts in Stevia leaves. In some cases, rubusoside is also present in extracts of Stevia leaves (Stevia rebaudiana Bertoni).

In some embodiments, the starting material for the enzymatic process is a terpene glycoside purified from either an extract of a Stevia rebaudiana Bertoni plants, or an extract of a Rubus suavissimus plants, or an extract of a Siraitis grosvenorii plant.

In some embodiments, the terpene glycoside starting material is selected from the group consisting of: stevioside, rebaudioside A, rebaudioside B, rebaudioside C, rebaudioside D, rebaudioside E, rebaudioside F, rebaudioside G, rebaudioside M, dulcoside A, steviolbioside, rubusoside, and any combinations thereof. In some further embodiments, the terpene glycoside starting material is selected from the group consisting of: stevioside, rebaudioside A, rebaudioside B, rebaudioside C, and rubusoside.

In some embodiments, the terpene glycoside starting material is a diterpene glycoside disclosed in U.S. Pat. No. 8,257,948. In some other embodiments, the terpene glycoside starting material is a terpene glycoside disclosed in PCT Publication No. WO 2017/089444. In some other embodiments, the terpene glycoside starting material is a terpene glycoside disclosed in PCT Publication No. WO 2013/019050. In some other embodiments, the terpene glycoside starting material is a terpene glycoside disclosed in European Patent Application Publication No. EP 3003058.

As used herein, the term “glycoside” refers to an organic compound to which one or more sugar units are covalently bound at one or more sites of the chemical structure.

As noted above, the reacting is carried out in an aqueous composition, such as an aqueous solution. In some such embodiments, the aqueous composition comprises deionized water. Alternatively, in some other embodiments, the aqueous composition comprises an aqueous solution of sodium acetate. In some further such embodiments, the concentration of the sodium acetate in the aqueous solution is 0.2M.

The aqueous composition can have any suitable pH, depending on the nature of the terpene glycoside, the reacting sugar, and the enzyme used. In some embodiments, the pH of the aqueous composition ranges from 4.0 to 7.0. In some further embodiments, the pH of the aqueous composition ranges from 4.0 to 6.0. In some further embodiments, the pH of the aqueous composition ranges from 4.0 to 5.0. In some other embodiments, the pH of the aqueous composition ranges from 5.0 to 7.0. In some other embodiments, the pH of the aqueous composition ranges from 6.0 to 7.0. In some embodiments, the pH of the aqueous composition is 4.0, or 4.1, or 4.2, or 4.3, or 4.4, or 4.5, or 4.6, or 4.7, or 4.8, or 4.9, or 5.0, or 5.1, or 5.2, or 5.3, or 5.4, or 5.5, or 5.6, or 5.7, or 5.8, or 5.9, or 6.0, or 6.1, or 6.2, or 6.3, or 6.4, or 6.5, or 6.6, or 6.7, or 6.8, or 6.9, or 7.0. In some embodiments, the pH of the aqueous composition is about 5.0.

In carrying out the reacting, the terpene glycoside can be added in any suitable concentration. In some embodiments, the terpene glycoside is present in the aqueous composition at a concentration ranging from 0.005 g/mL to 0.5 g/mL. In some further embodiments, the terpene glycoside is present in the aqueous composition at a concentration ranging from 0.05 g/mL to 0.25 g/mL. In some further embodiments, the terpene glycoside is added to the aqueous solution at an amount from 0.1 to 0.2 g/ml.

As noted above, the methods disclosed herein employ an α-glucosyl sugar compound. As used herein, the term “α-glucosyl sugar compound” refers to a saccharide containing at least one alpha-glucosyl residue. In some non-limiting embodiments, the α-glucosyl sugar compound is selected from the group consisting of: maltose, maltotriose, maltotetraose, partial hyrdrolysates of statch, maltodextrin, glucose, sucrose, and combinations thereof. In some embodiments, the α-glucosyl sugar compound is maltodextrin. In some embodiments, the α-glucosyl sugar compound is maltose. In some embodiments, the α-glucosyl sugar compound is selected from the α-glucosyl sugar compounds disclosed in Great Britain Patent Publication No. 2027432.

The α-glucosyl sugar compounds contemplated for use herein can have any suitable value for their dextrose equivalent. In some embodiments, the α-glucosyl sugar compound has a dextrose equivalent ranging from 10 to 25, or from 12 to 20. In some embodiments, the α-glucosyl sugar compound has a dextrose equivalent of about 18.

In the methods contemplated herein, the α-glucosyl sugar compound can have any suitable concentration in the aqueous composition. In some embodiments, the concentration of the α-glucosyl sugar compound in the aqueous composition ranges from 10 percent by weight (w/w) to 40 percent by weight, or from 20 percent by weight to 30 percent by weight. In some embodiments, the concentration of the α-glucosyl sugar compound in the aqueous composition ranges from 0.005 g/mL to 0.5 g/mL. In some embodiments, the concentration of the α-glucosyl sugar compound in the aqueous composition is about 0.2 g/mL.

In the methods contemplated herein, the terpene glycoside and the α-glucosyl sugar can be present in the aqueous composition in any suitable ratio relative to each other. In some embodiments, the ratio (w/w) of the terpene glycoside to the α-glucosyl sugar compound in the aqueous composition ranges from 100:1 to 1:100, or from 10:1 to 1:10. In some embodiments, the ratio (w/w) of the terpene glycoside to the α-glucosyl sugar compound in the aqueous composition is about 1:1.

In the methods disclosed herein, the transglucosidase performs a transglucosylation reaction, thereby generating a glucosylated terpene glycoside having a single α-1,6 glucosidic bond. Alternatively, in some embodiments thereof, the transglucosidase performs a transglucosylation reaction, thereby generating a glucosylated terpene glycoside having one or more glucose residues covalently attached to the terpene glycoside via an α-1,6 glucosidic bond. In some embodiments, the number of glucose residues that are added to the terpene glycoside may be controlled by parameters such as, for example, the duration of the reaction, the temperature of the reaction, the concentration of the terpene glycoside, the concentration of the α-glucosyl sugar compound, and the like.

In some embodiments, the transglucosidase performs the transglucosylation reaction, using maltose or maltodextrin as substrates, thereby generating a glucosylated terpene glycoside wherein either a single maltose residue is added to the terpene glycoside via an α-1,6 glucosidic bond, or, alternatively, two glucose units are added to the terpene glycoside via an α-1,6 glucosidic bond.

The transglucosidase may be provided in any suitable way. In some embodiments, the transglucosidase is in a form of cell-free culture broth, concentrated liquid cell-free culture broth, spray dried or freeze dried cell-free culture broth, or high purity protein. In some embodiments, free and immobilized enzyme preparations are used. In some embodiments, the transglucosidase is Aspergillus niger glucosyltransferase. In certain embodiments thereof, the activity of transglucosidase may be determined according to the procedure described in Hale W. S., Rawlins L. C. (1951) Amylase of Bacillus macerans. Cereal Chem. 28, 49-58.

In the methods contemplated by the present disclosure, the transglucosidase is present in the aqueous composition in any suitable concentration. In some embodiments, the transglucosidase is present in the mixture at an amount ranging from 0.2 to 0.4 units per gram of the α-glucosyl sugar compound.

In the methods contemplated herein, the enzyme can be present in any suitable ratio relative to the terpene glycoside. In some embodiments, the ratio of the amount of enzyme in wt % to the amount of terpene glycoside in wt % ranges from 1:1000 to 1:1. In some further embodiments, the ratio of the amount of enzyme in wt % to the amount of terpene glycoside in wt % ranges from 1:100 to 1:2. In some embodiments, the ratio of the amount of enzyme in wt % to the amount of terpene glycoside in wt % ranges from 1:10 to 1:5.

In the methods contemplated herein, the reacting can be carried out in any suitable manner. In some embodiments, the aqueous composition is incubated for a time and temperature sufficient to generate the glucosylated terpene glycoside. In some such embodiments, the temperature ranges from 30° C. to 90° C., or from 70° C. to 90° C. In some embodiments, the temperature is about 60° C.

The reacting may be allowed to proceed for any suitable length of time. In some embodiments, the duration is 24 hours or greater, such as 24-48 hours. Alternatively, in some other embodiments, the duration is 24 hours or less, such as 4 to 24 hours. In some embodiments, the duration is 24 hours, or 23 hours, or 22 hours, or 21 hours, or 20 hours, or 19 hours, or 18 hours, or 17 hours, or 16 hours, or 15 hours, or 14 hours, or 13 hours, or 12 hours, or 11 hours, or 10 hours, or 9 hours, or 8 hours, or 7 hours, or 6 hours, or 5 hours, or 4 hours, or 3 hours, or 2 hours, or 1 hour. In some embodiments, the duration is about 24 hours.

In some embodiments, after the reacting step, the mixture containing the glucosylated terpene glycoside is treated further. Such further treatment may include, for example, an inactivation step or a purification step, wherein the glucosylated terpene glycoside is isolated or purified. Non-limiting examples of the purification step include, for example, enrichment, isolation, or purification of the glucosylated terpene glycoside, or the removal of solids from the reaction mixture. For example, solids may be removed from the reaction mixture by means such as filtration, centrifugation, or other techniques known to those skilled in the art. In another example, carbohydrates may be removed from the mixture using adsorption resins, precipitation, or other techniques known to those skilled in the art.

In some embodiments, the further treatment comprises inactivating the transglucosidase. In one non-limiting example, the transglucosidase is inactivated by the application of heat. In some embodiments, the transglucosidase is inactivated by heating the reaction mixture to a temperature sufficient to inactivate the transglucosidase. In some embodiments, the temperature is at least 100° C.

U.S. Pat. No. 8,257,948 discloses some examples of purification steps that may be utilized in some aspects of the present disclosure to isolate or purify the glucosylated terpene glycoside. PCT Publication No. WO 2017/089444 discloses other examples of purification steps that may be utilized in some aspects of the present disclosure to isolate or purify the glucosylated terpene glycoside. PCT Publication No. WO 2013/019050 discloses other examples of purification steps that may be utilized in some aspects of the present disclosure to isolate or purify the glucosylated terpene glycoside. European Patent Application Publication No. 3003058 discloses other examples of purification steps that may be utilized in some aspects of the present disclosure to isolate or purify the glucosylated terpene glycoside. U.S. Pat. No. 8,257,948 discloses some examples of inactivation steps that may be utilized in some aspects of the present disclosure. PCT Publication No.

WO 2017/089444 discloses other examples of inactivation steps that may be utilized in some aspects of the present disclosure.

### The Glucosylated Terpene Glycosides

In some embodiments, the glucosylated terpene glycoside is selected from the group consisting of: mono α-1,6 glucosylated stevioside, mono α-1,6 glucosylated rebaudioside A, mono α-1,6 glucosylated rebaudioside B, mono α-1,6 glucosylated rebaudioside C, mono α-1,6 glucosylated rebaudioside D, mono α-1,6 glucosylated rebaudioside E, mono α-1,6 glucosylated rebaudioside F, mono α-1,6 glucosylated rebaudioside G, mono α-1,6 glucosylated rebaudioside M, mono α-1,6 glucosylated dulcoside A, mono α-1,6 glucosylated steviolbioside, mono α-1,6 glucosylated rubusoside, and any combinations thereof.

In some embodiments, the glucosylated terpene glycoside is selected from the group consisting of: di α-1,6 glucosylated stevioside, di α-1,6 glucosylated rebaudioside A, di α-1,6 glucosylated rebaudioside B, di α-1,6 glucosylated rebaudioside C, di α-1,6 glucosylated rebaudioside D, di α-1,6 glucosylated rebaudioside E, di α-1,6 glucosylated rebaudioside F, di α-1,6 glucosylated rebaudioside G, di α-1,6 glucosylated rebaudioside M, di α-1,6 glucosylated dulcoside A, di α-1,6 glucosylated steviolbioside, di α-1,6 glucosylated rubusoside, and any combinations thereof.

In some embodiments, the glucosylated terpene glycoside is selected from the group consisting of: mono α-1,6 maltosylated stevioside, mono α-1,6 maltosylated rebaudioside A, mono α-1,6 maltosylated rebaudioside B, mono α-1,6 maltosylated rebaudioside C, mono α-1,6 maltosylated rebaudioside D, mono α-1,6 maltosylated rebaudioside E, mono α-1,6 maltosylated rebaudioside F, mono α-1,6 maltosylated rebaudioside G, mono α-1,6 maltosylated rebaudioside M, mono α-1,6 maltosylated dulcoside A, mono α-1,6 maltosylated steviolbioside, mono α-1,6 maltosylated rubusoside, and any combinations thereof.

In some aspects, the disclosure provides a compound of formula I:

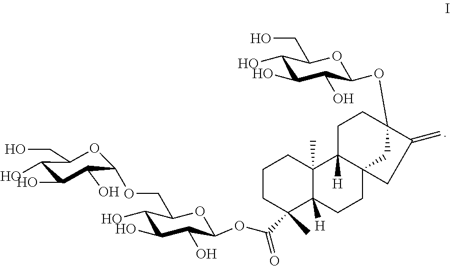

In some aspects, the disclosure provides a compound of formula II:

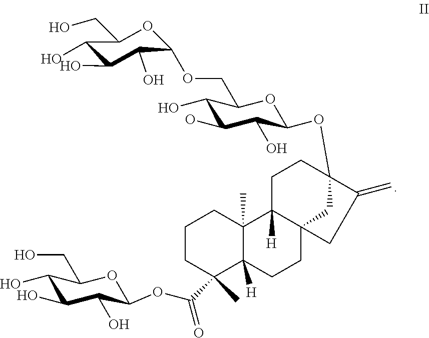

In some aspects, the disclosure provides a compound of formula III:

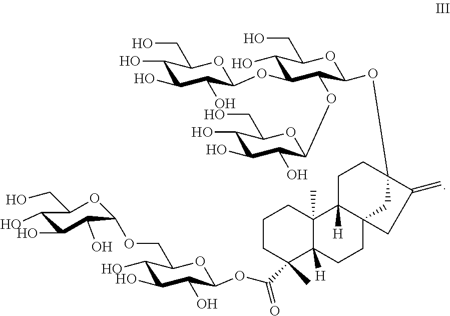

In some aspects, the disclosure provides a compound of formula IV:

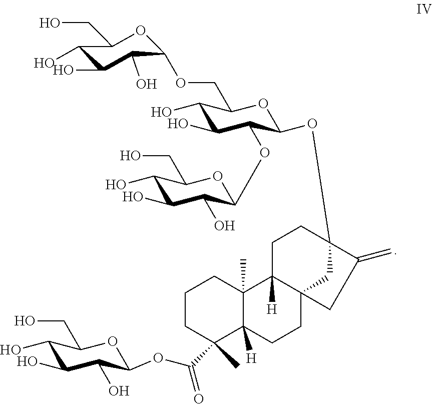

In some aspects, the disclosure provides a compound of formula V:

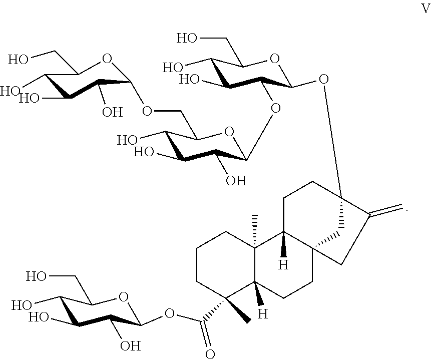

In some aspects, the disclosure provides a compound of formula VI:

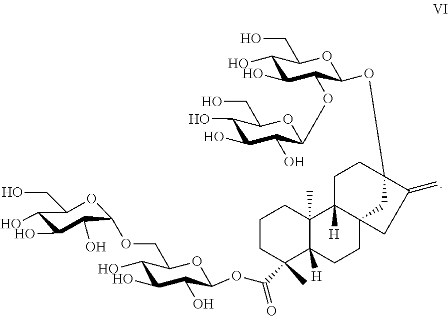

In some aspects, the disclosure provides a compound of formula VII:

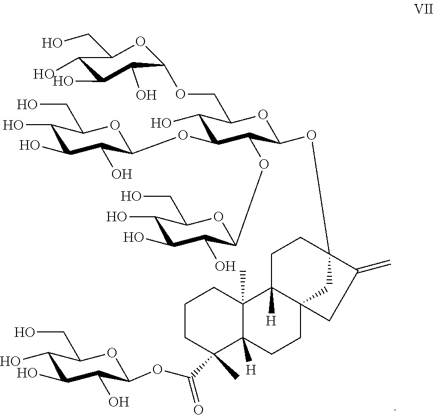

In some aspects, the disclosure provides a compound of formula:

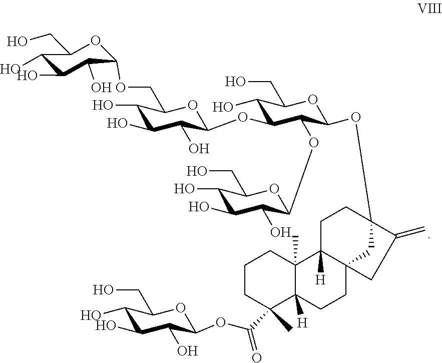

In some aspects, the disclosure provides a compound of formula IX:

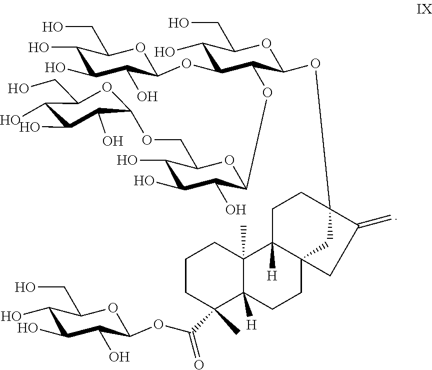

One at least one aspect disclosed herein, the disclosure provides a composition comprising at least one glucosylated terpene glycoside selected from the group consisting of: mono α-1,6 glucosylated stevioside, mono α-1,6 glucosylated rebaudioside A, mono α-1,6 glucosylated rebaudioside B, mono α-1,6 glucosylated rebaudioside C, mono α-1,6 glucosylated rebaudioside D, mono α-1,6 glucosylated rebaudioside E, mono α-1,6 glucosylated rebaudioside F, mono α-1,6 glucosylated rebaudioside G, mono α-1,6 glucosylated rebaudioside M, mono α-1,6 glucosylated dulcoside A, mono α-1,6 glucosylated steviolbioside, mono α-1,6 glucosylated rubusoside, di α-1,6 glucosylated stevioside, di α-1,6 glucosylated rebaudioside A, di α-1,6 glucosylated rebaudioside B, di α-1,6 glucosylated rebaudioside C, di α-1,6 glucosylated rebaudioside D, di α-1,6 glucosylated rebaudioside E, di α-1,6 glucosylated rebaudioside F, di α-1,6 glucosylated rebaudioside G, di α-1,6 glucosylated rebaudioside M, di α-1,6 glucosylated dulcoside A, di α-1,6 glucosylated steviolbioside, di α-1,6 glucosylated rubusoside, mono α-1,6 maltosylated stevioside, mono α-1,6 maltosylated rebaudioside A, mono α-1,6 maltosylated rebaudioside B, mono α-1,6 maltosylated rebaudioside C, mono α-1,6 maltosylated rebaudioside D, mono α-1,6 maltosylated rebaudioside E, mono α-1,6 maltosylated rebaudioside F, mono α-1,6 maltosylated rebaudioside G, mono α-1,6 maltosylated rebaudioside M, mono α-1,6 maltosylated dulcoside A, mono α-1,6 maltosylated steviolbioside, and mono α-1,6 glucosylated rubusoside.

In at least another aspect, the disclosure provides a composition comprising at least one glucosylated terpene glycoside selected from the group consisting of: a compound of formula I, a compound of formula II, a compound of formula III, a compound of formula IV, a compound of formula V, a compound of formula VI, a compound of formula VII, a compound of formula VIII, and a compound of formula IX. In a further embodiment thereof, the disclosure provides a composition comprising a compound of formula I, a compound of formula II, a compound of formula III, and a compound of formula VIII.

### Sweeteners and/or Sweetness Enhancers

The glucosylated terpene glycosides described herein can be used as sweetness enhancers, flavor enhancers, or sweeteners in various flavored articles. In some embodiments thereof, the disclosure provides uses of such glucosylated terpene glycosides to confer, enhance, improve, or modify a sweet taste of a flavored article.

In some embodiments, the concentration effective to confer, enhance, improve, or modify the sweet taste of the flavored article ranges from 1 ppm to 1000 ppm, or from 1 ppm to 500 ppm, or from 1 ppm to 200 ppm, or from 1 ppm to 100 ppm, or from 1 ppm to 75 ppm, or from 1 ppm to 50 ppm. In some embodiments, the concentration effective to confer, enhance, improve, or modify the sweet taste of the flavored article is about 40 ppm. In some aspects, the amount effective to confer, enhance, improve, or modify the sweet taste of the flavored article is less than 40 ppm. In some aspects, the amount effective to confer, enhance, improve, or modify the sweet taste of the flavored article is greater than 40 ppm. In some aspects, the amount effective to confer, enhance, improve, or modify the sweet taste of the flavored article is between 0 and 1000 ppm.

In some embodiments, the composition comprises a glucosylated terpene glycoside (according to any of the foregoing embodiments) and, a foodstuff base. Non-limiting examples of suitable foodstuffs, e.g. foods or beverages are also provided herein. For the purpose of the present disclosure, “foodstuff base” means an edible product, e.g. a food or a beverage. Therefore, a flavored article provided herein comprises the functional formulation, as well as optionally additional benefit agents, corresponding to a desired edible product, e.g. a savory cube, and a flavor effective amount of the at least one glucosylated terpene glycosides described herein.

The compositions can include any suitable sweeteners or combination of sweeteners. In some embodiments, the sweetener is a common saccharide sweeteners, such as sucrose, fructose, glucose, and sweetener compositions comprising natural sugars, such as corn syrup (including high fructose corn syrup) or other syrups or sweetener concentrates derived from natural fruit and vegetable sources. In some embodiments, the sweetener is sucrose, fructose, or a combination thereof. In some embodiments, the sweetener is sucrose. In some other embodiments, the sweetener is selected from rare natural sugars including D-allose,

D-psicose, L-ribose, D-tagatose, L-glucose, L-fucose, L-arbinose, D-turanose, and

D-leucrose. In some embodiments, the sweetener is selected from semi-synthetic “sugar alcohol” sweeteners such as erythritol, isomalt, lactitol, mannitol, sorbitol, xylitol, maltodextrin, and the like. In some embodiments, the sweetener is selected from artificial sweeteners such as aspartame, saccharin, acesulfame-K, cyclamate, sucralose, and alitame. In some embodiments, the sweetener is selected from the group consisting of cyclamic acid, mogroside, tagatose, maltose, galactose, mannose, sucrose, fructose, lactose, neotame and other aspartame derivatives, glucose, D-tryptophan, glycine, maltitol, lactitol, isomalt, hydrogenated glucose syrup (HGS), hydrogenated starch hydrolyzate (HSH), stevioside, rebaudioside A and other sweet Stevia-based glycosides, carrelame and other guanidine-based sweeteners. In some embodiments, the sweetener is a combination of two or more of the sweeteners set forth in this paragraph. In some embodiments, the sweetener may combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a sugar. In some embodiments, the sugar is cane sugar. In some embodiments, the sugar is beet sugar. In some embodiments, the sugar may be sucrose, fructose, glucose or combinations thereof. In some embodiments, the sugar may be sucrose. In some embodiments, the sugar may be a combination of fructose and glucose.

The sweetener can also include, for example, sweetener compositions comprising one or more natural or synthetic carbohydrate, such as corn syrup, high fructose corn syrup, high maltose corn syrup, glucose syrup, sucralose syrup, hydrogenated glucose syrup (HGS), hydrogenated starch hydrolyzate (HSH), or other syrups or sweetener concentrates derived from natural fruit and vegetable sources, or semi-synthetic “sugar alcohol” sweeteners such as polyols. Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, isomaltulose, maltodextrin, and the like, and sugar alcohols or any other carbohydrates or combinations thereof capable of being reduced which do not adversely affect taste.

The sweetener may be a natural or synthetic sweetener that includes, but is not limited to, agave inulin, agave nectar, agave syrup, amazake, brazzein, brown rice syrup, coconut crystals, coconut sugars, coconut syrup, date sugar, fructans (also referred to as inulin fiber, fructo-oligosaccharides, or oligo-fructose), green Stevia powder, Stevia rebaudiana, rebaudioside A, rebaudioside B, rebaudioside C, rebaudioside D, rebaudioside E, rebaudioside F, rebaudioside I, rebaudioside H, rebaudioside L, rebaudioside K, rebaudioside J, rebaudioside N, rebaudioside O, rebaudioside M and other sweet Stevia-based glycosides, stevioside, stevioside extracts, honey, Jerusalem artichoke syrup, licorice root, luo han guo (fruit, powder, or extracts), lucuma (fruit, powder, or extracts), maple sap (including, for example, sap extracted from Acer saccharum, Acer nigrum, Acer rubrum, Acer saccharinum, Acer platanoides, Acer negundo, Acer macrophyllum, Acer grandidentatum, Acer glabrum, Acer mono), maple syrup, maple sugar, walnut sap (including, for example, sap extracted from Juglans cinerea, Juglans nigra, Juglans ailatifolia, Juglans regia), birch sap (including, for example, sap extracted from Betula papyrifera, Betula alleghaniensis, Betula lenta, Betula nigra, Betula populifolia, Betula pendula), sycamore sap (such as, for example, sap extracted from Platanus occidentalis), ironwood sap (such as, for example, sap extracted from Ostrya virginiana), mascobado, molasses (such as, for example, blackstrap molasses), molasses sugar, monatin, monellin, cane sugar (also referred to as natural sugar, unrefined cane sugar, or sucrose), palm sugar, panocha, piloncillo, rapadura, raw sugar, rice syrup, sorghum, sorghum syrup, cassava syrup (also referred to as tapioca syrup), thaumatin, yacon root, malt syrup, barley malt syrup, barley malt powder, beet sugar, cane sugar, crystalline juice crystals, caramel, carbitol, carob syrup, castor sugar, hydrogenated starch hydrolates, hydrolyzed can juice, hydrolyzed starch, invert sugar, anethole, arabinogalactan, arrope, syrup, P-4000, acesulfame potassium (also referred to as acesulfame K or ace-K), alitame (also referred to as aclame), advantame, aspartame, baiyunoside, neotame, benzamide derivatives, bernadame, canderel, carrelame and other guanidine-based sweeteners, vegetable fiber, corn sugar, coupling sugars, curculin, cyclamates, cyclocarioside I, demerara, dextran, dextrin, diastatic malt, dulcin, sucrol, valzin, dulcoside A, dulcoside B, emulin, enoxolone, maltodextrin, saccharin, estragole, ethyl maltol, glucin, gluconic acid, glucono-lactone, glucosamine, glucoronic acid, glycerol, glycine, glycyphillin, glycyrrhizin, golden sugar, yellow sugar, golden syrup, granulated sugar, gynostemma, hernandulcin, isomerized liquid sugars, jallab, chicory root dietary fiber, kynurenine derivatives (including N′-formyl-kynurenine, N′-acetyl-kynurenine, 6-chloro-kynurenine), galactitol, litesse, ligicane, lycasin, lugduname, guanidine, falernum, mabinlin I, mabinlin II, maltol, maltisorb, maltodextrin, maltotriol, mannosamine, miraculin, mizuame, mogrosides (including, for example, mogroside IV, mogroside V, and neomogroside), mukurozioside, nano sugar, naringin dihydrochalcone, neohesperidine dihydrochalcone, nib sugar, nigero-oligosaccharide, norbu, orgeat syrup, osladin, pekmez, pentadin, periandrin I, perillaldehyde, perillartine, petphyllum, phenylalanine, phlomisoside I, phlorodizin, phyllodulcin, polyglycitol syrups, polypodoside A, pterocaryoside A, pterocaryoside B, rebiana, refiners syrup, rub syrup, rubusoside, selligueain A, shugr, siamenoside I, Siraitia grosvenorii, soybean oligosaccharide, Splenda, SRI oxime V, steviol glycoside, steviolbioside, stevioside, strogins 1, 2, and 4, sucronic acid, sucrononate, sugar, suosan, phloridzin, superaspartame, tetrasaccharide, threitol, treacle, trilobtain, tryptophan and derivatives (6-trifluoromethyl-tryptophan, 6-chloro-D-tryptophan), vanilla sugar, volemitol, birch syrup, aspartame-acesulfame, assugrin, and combinations or blends of any two or more thereof.

In still other embodiments, the sweetener can be a chemically or enzymatically modified natural high potency sweetener. Modified natural high potency sweeteners include glycosylated natural high potency sweetener such as glucosyl-, galactosyl-, or fructosyl-derivatives containing 1-50 glycosidic residues. Glycosylated natural high potency sweeteners may be prepared by enzymatic transglycosylation reaction catalyzed by various enzymes possessing transglycosylating activity. In some embodiments, the modified sweetener can be substituted or unsubstituted.

Additional sweeteners also include combinations of any two or more of any of the aforementioned sweeteners. In some embodiments, the sweetener may comprise combinations of two, three, four or five sweeteners as disclosed herein. In some embodiments, the sweetener may be a sugar. In some embodiments, the sweetener may be a combination of one or more sugars and other natural and artificial sweeteners. In some embodiments, the sweetener is a caloric sweetener, such as sucrose, fructose, xylitol, erythritol, or combinations thereof. In some embodiments, the ingestible compositions are free (or, in some embodiments) substantially free of Stevia-derived sweeteners, such as steviol glycosides, glucosylated steviol glycosides, or rebaudiosides. For examole, in some embodiments, the ingestible compositions are either free of Stevia-derived sweeteners or comprise Stevia-derived sweeteners in a concentration of no more than 1000 ppm, or no more than 500 ppm, or no more than 200 ppm, or no more than 100 ppm, or no more than 50 ppm, or no more than 20 ppm, or no more than 10 ppm, or no more than 5 ppm, or no more than 3 ppm, or no more than 1 ppm.

The ingestible compositions disclosed herein can be formulated into any kind of foodstuffs. The compositions and methods provided herein have use in food or beverage products. When the food product is a particulate or powdery food, the dry particles may easily be added thereto by dry-mixing. Typical food products are selected from the group consisting of an instant soup or sauce, a breakfast cereal, a powdered milk, a baby food, a powdered drink, a powdered chocolate drink, a spread, a powdered cereal drink, a chewing gum, an effervescent tablet, a cereal bar, and a chocolate bar. The powdered foods or drinks may be intended to be consumed after reconstitution of the product with water, milk and/or a juice, or another aqueous liquid.

The food product may be selected from the group consisting of condiments, baked goods, powdery food, bakery filings and fluid dairy products. Condiments include, without limitation, ketchup, mayonnaise, salad dressing, Worcestershire sauce, fruit-flavored sauce, chocolate sauce, tomato sauce, chili sauce, and mustard.

Baked goods include, without limitation, cakes, cookies, pastries, breads, donuts and the like.

Bakery fillings include, without limitation, low or neutral pH fillings, high, medium or low solids fillings, fruit or milk based (pudding type or mousse type) fillings, hot or cold make-up fillings and nonfat to full-fat fillings.

Fluid dairy products include, without limitation, non-frozen, partially frozen and frozen fluid dairy products such as, for example, milks, ice creams, sorbets and yogurts. Beverage products include, without limitation, carbonated soft drinks, including cola, lemon-lime, root beer, heavy citrus (“dew type”), fruit flavored and cream sodas; powdered soft drinks, as well as liquid concentrates such as fountain syrups and cordials; coffee and coffee-based drinks, coffee substitutes and cereal-based beverages; teas, including dry mix products as well as ready-to-drink teas (herbal and tealeaf based); fruit and vegetable juices and juice flavored beverages as well as juice drinks, nectars, concentrates, punches and “ades”; sweetened and flavored waters, both carbonated and still; sport/energy/health drinks; alcoholic beverages plus alcohol-free and other low-alcohol products including beer and malt beverages, cider, and wines (still, sparkling, fortified wines and wine coolers); other beverages processed with heating (infusions, pasteurization, ultra-high temperature, ohmic heating or commercial aseptic sterilization) and hot-filled packaging; and cold-filled products made through filtration or other preservation techniques.

The nature and type of the constituents of the foodstuffs or beverages do not warrant a more detailed description here, which in any case would not be exhaustive, the skilled person being able to select them on the basis of his general knowledge and according to the nature of the product.

The proportions in which the at least one glucosylated terpene glycoside having a single α-1,6 glucosidic bond described herein can be incorporated into the various aforementioned articles or compositions vary within a wide range of values. These values are dependent on the nature of the article to be flavored and on the desired organoleptic effect as well as the nature of the co-ingredients in a given base when the compounds according to the invention are mixed with flavoring co-ingredients, solvents or additives commonly used in the art.

In the case of flavoring compositions, typical concentrations are in the order of 0.0001% to 1% by weight, or even more, of the at least one glucosylated terpene glycoside described herein based on the weight of the consumer product into which they are incorporated. Concentrations lower than these, such as in the order of 0.001% to 0.5% by weight, can be used when the at least one glucosylated terpene glycoside having a single α-1,6 glucosidic bond described herein is incorporated into flavored articles, percentage being relative to the weight of the article.

The present invention is best illustrated but is not limited to the following examples.

### EXAMPLES

### Example 1: Generation of Mono α-1,6-glucosylated Stevioside Compounds (Compounds I and II) Using Rubusoside and Maltodextrin as Starting Materials by a Method According to Some Aspects Presented Herein

Rubusoside (2 g) and corn maltodextrin (2 g) having a dextrose equivalent (DE) of 18 were dissolved in 10 ml NaOAc—HOAc (pH=6.0, 0.2 M, 10 mL) buffer or deionized water at room temperature. Subsequently, 100 μl Aspergillus niger glucosyltransferase (Transglucosidase L; Amano Enzymes, Nagoya, Japan) was added to the mixture. The mixture containing the enzyme was then heated to 60° C., and the mixture containing the enzyme was incubated at 60° C. for 24 hours to allow the transglucosidation reaction to proceed, thereby generating glucosylated terpene glycosides having a single α-1,6 glucosidic bond. The reaction was terminated by inactivating the transglucosidase by incubating the reaction mixture at 100° C. for 30 minutes.

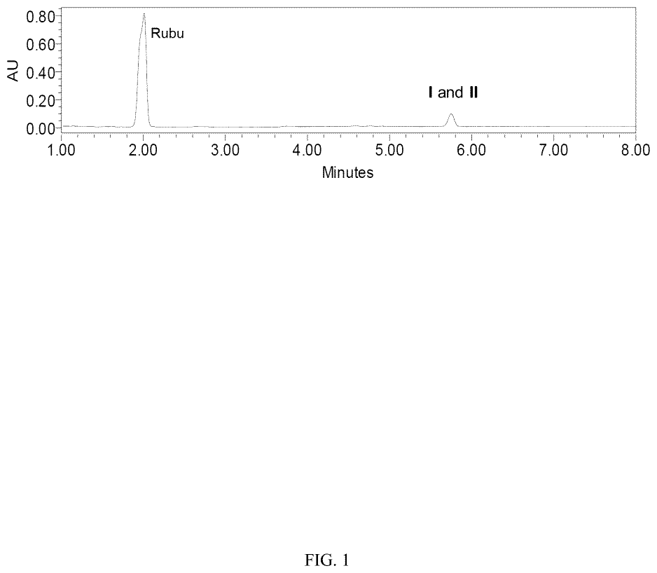

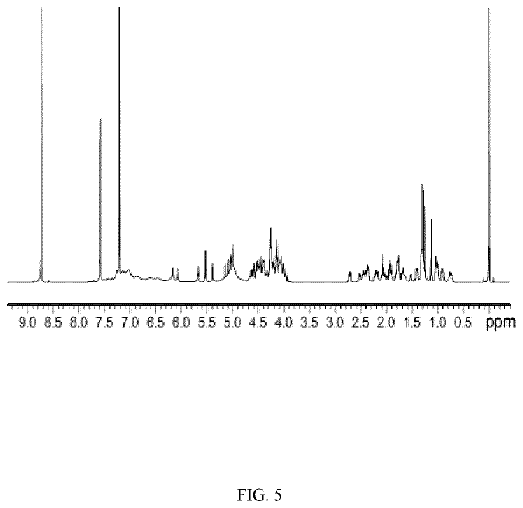

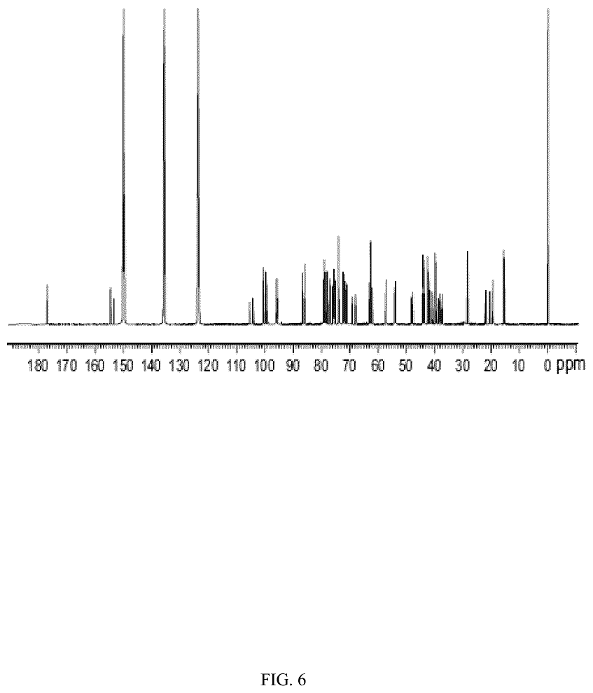

The resulting reaction mixture was analyzed by UPLC-UV, and a mixture containing glucosylated terpene glycosides having a single α-1,6 glucosidic bond was identified (See FIG. 1). The identified mixture was purified via a prep-LC. The compounds within the mixture were identified as a compound of formula I and a compound of formula II. FIGS. 5 and 6 show ¹H and ¹³C NMR spectra, respectively, for the mixture of the two compounds.

### Example 2: Generation of Mono α-1,6-glucosylated Stevioside Compounds (Compounds I and II) Using Rubusoside and Maltose as Starting Materials by a Method According to Some Aspects Presented Herein

Rubusoside (2 g) and maltose (2 g) were dissolved in 10 ml NaOAc—HOAc (pH=6.0, 0.2 M, 10 mL) buffer or deionized water at room temperature. Subsequently, 100 μL Aspergillus niger glucosyltransferase (Transglucosidase L; Amano Enzymes, Nagoya, Japan) was added to the mixture. The mixture containing the enzyme was then heated to 60° C., and the mixture containing the enzyme was incubated at 60° C. for 24 hours to allow the transglucosidation reaction to proceed, thereby generating glucosylated terpene glycosides having a single α-1,6 glucosidic bond. The reaction was terminated by inactivating the transglucosidase by incubating the reaction mixture at 100° C. for 30 minutes.

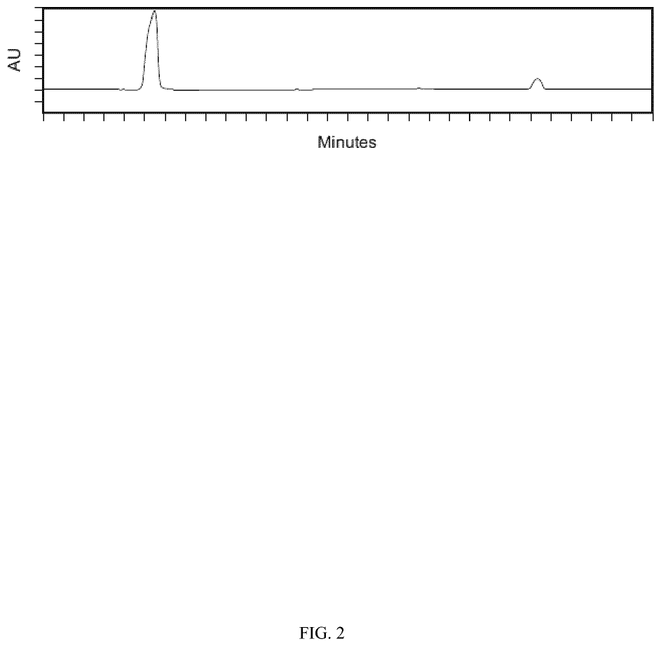

The resulting reaction mixture was analyzed by UPLC-UV, and a mixture containing glucosylated terpene glycosides having a single α-1,6 glucosidic bond was identified (See FIG. 2). The identified mixture was purified via a prep-LC. The compounds within the mixture were identified as a compound of formula I and a compound of formula II.

### Example 3: Generation of Mono α-1,6-glucosylated Rebaudioside A (Compound III) Using Rebaudioside as a Starting Material by a Method According to Some Aspects Presented Herein

Rebaudioside A (2 g) and corn maltodextrin (2 g) having a dextrose equivalent (DE) of 18 were dissolved in 10 ml deionized water at room temperature. Subsequently, 100 μl Aspergillus niger glucosyltransferase (Transglucosidase L; Amano Enzymes, Nagoya, Japan) was added to the mixture. The mixture containing the enzyme was then heated to 60° C., and the mixture containing the enzyme was incubated at 60° C. for 24 hours to allow the transglucosidation reaction to proceed, thereby generating glucosylated terpene glycosides having a single α-1,6 glucosidic bond. The reaction was terminated by inactivating the transglucosidase by incubating the reaction mixture at 100° C. for 30 minutes.

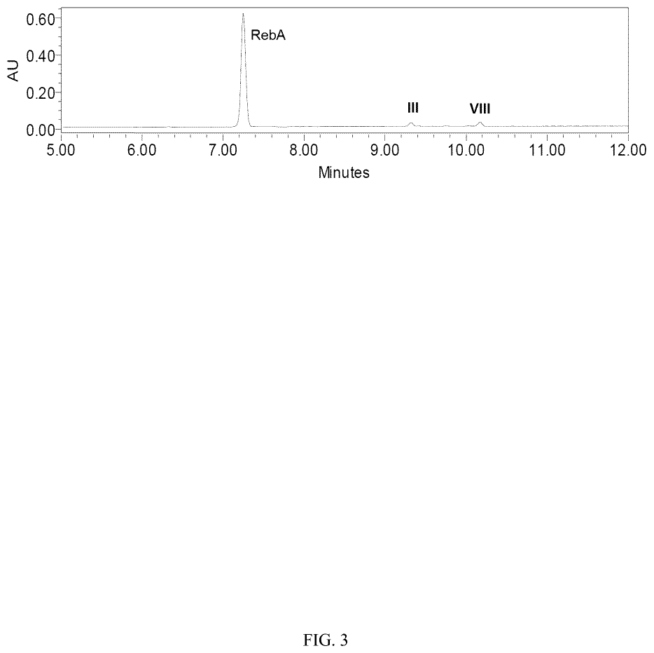

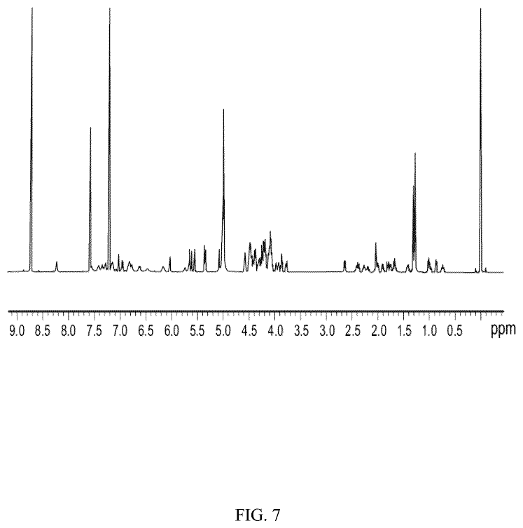

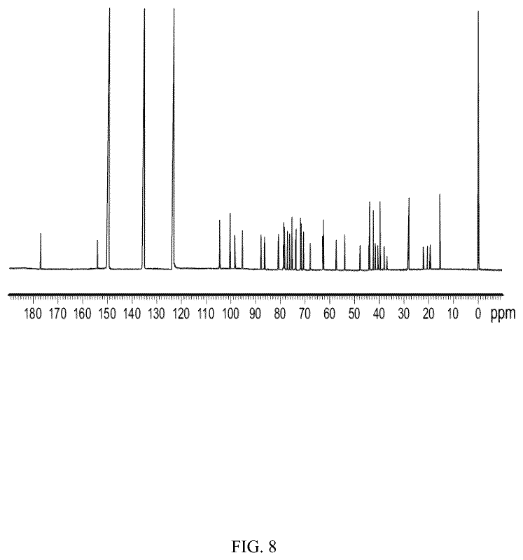

The resulting reaction mixture was analyzed by UPLC-UV, and glucosylated terpene glycosides having a single α-1,6 glucosidic bond were identified (See FIG. 3). The identified mixture was purified via a prep-LC. The glucosylated terpene glycosides having a single α-1,6 glucosidic bond were identified as a compound of formula III and a compound of formula VIII. FIGS. 7 and 8 show ¹H and ¹³C NMR spectra, respectively, for the compound of formula III.

### Example 4: Generation of Mono α-1,6-glucosylated Rebaudioside A (Compound III) Using Rebaudioside as a Starting Material by a Method According to Some Aspects Presented Herein

A composition of steviol glycoside (90% w/w) (1 g) and corn maltodextrin (1 g) having a dextrose equivalent (DE) of 18 were dissolved in 5 ml deionized water at room temperature. Subsequently, 100 μl Aspergillus niger glucosyltransferase (Transglucosidase L; Amano Enzymes, Nagoya, Japan) was added to the mixture. The mixture containing the enzyme was then heated to 60° C., and the mixture containing the enzyme was incubated at 60° C. for 24 hours to allow the transglucosidation reaction to proceed, thereby generating glucosylated terpene glycosides having a single α-1,6 glucosidic bond. The reaction was terminated by inactivating the transglucosidase by incubating the reaction mixture at 100° C. for 30 minutes.

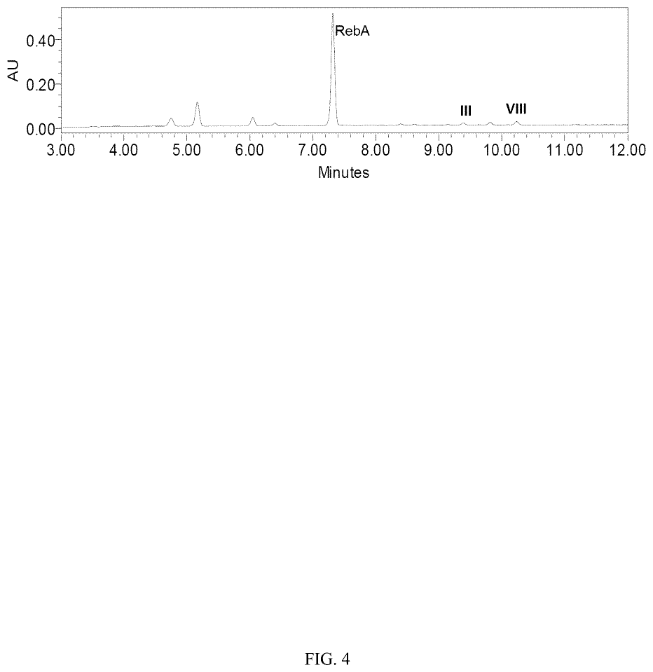

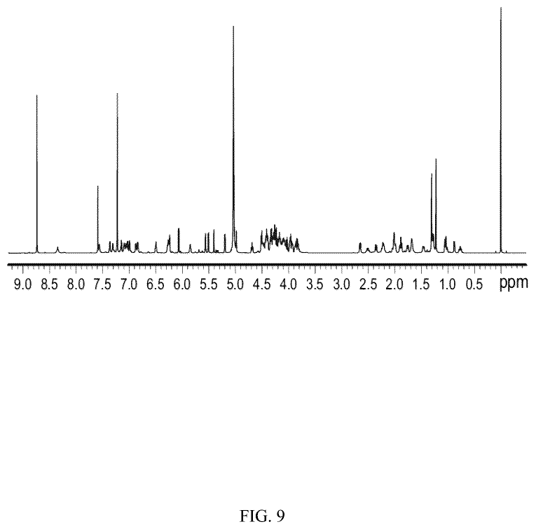

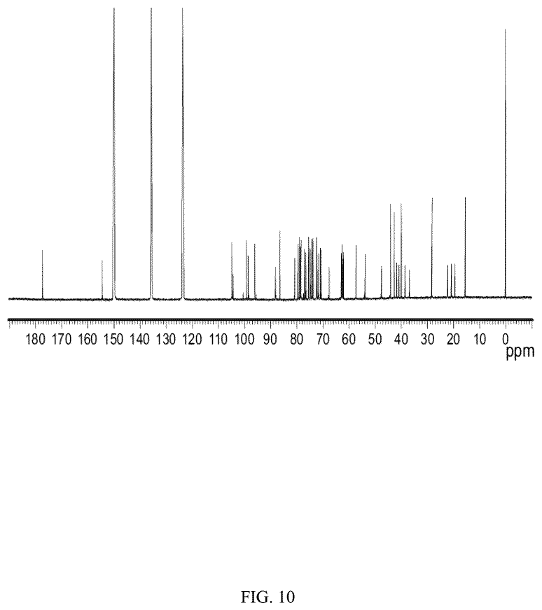

The resulting reaction mixture was analyzed by UPLC-UV, and glucosylated terpene glycosides having a single α-1,6 glucosidic bond were identified (See FIG. 4). The identified mixture was purified via a prep-LC. The glucosylated terpene glycosides having a single α-1,6 glucosidic bond were identified as a compound of formula III and a compound of formula VIII. FIGS. 9 and 10 show ¹H and ¹³C NMR spectra, respectively, for the compound of formula VIII.

### Example 5: Sensory Properties of a Composition Comprising the Mono α-1,6-glucosylated Stevioside a Compound of Formula I and a Compound of Formula II

A composition comprising Compounds I and II was generated according to the methods described in Example 1. The composition was dissolved in either (i) water, or (ii) a 2% w/w sucrose solution, wherein the final concentration of the composition in solution was 40 ppm. Corresponding control solutions of either (i) 1.5%, or (ii) 2% w/w sucrose were also generated. A panel of 6 experts evaluated the difference between solution of the test composition and the sucrose solutions, using the 3-Alternative Forced Choice (3-AFC) and sweet intensity scale method. All samples were tested in blind in a random order.

The sweet intensity of the solution containing 40 ppm of the composition comprising Compounds I and II was equal to 1.5% sucrose solution based on 3-AFC method. This suggests the sweet intensity of the solution containing 40 ppm of the composition comprising Compounds I and II in water solution was low, almost at the threshold of sweet. Adding 40 ppm of the composition comprising a compound of formula I and a compound of formula II into 2% sucrose solution significantly increased sweet intensity from 3 to 5 based on sweet intensity scale (scale range: 0-10). This suggests that the solution containing 40 ppm of the composition comprising Compounds I and II enhances the sweet intensity of sucrose from barely sweet to moderate sweet. Consequently, the solution containing 40 ppm of the composition comprising Compounds I and II was an effective sweet enhancement for sucrose.

### Example 6: Sensory Properties of a Composition Comprising the at Least One Mono α-1,6-glucosylated Terpene Glycosides Presented Herein

A composition comprising the at least one glucosylated terpene glucosides was generated according to the methods described in the preceding examples. The compounds were dissolved in water (alternatively, the compounds could also be combined into a 2% (w/w) or 4% (w/w) sucrose solution or 7% (w/w) inverted sugar plus 0.15% citric acid (w/w) solution), wherein the final concentration of the composition in solution may range from 1 to 1000 ppm. A panel of taste experts evaluated the solutions.

A mixture comprising the compound of formula I and the compound of formula II: A moderate sweet taste, approximately 100-200 times sweeter than sucrose was perceived when a samples of the purified compound was tasted at a concentration of 500 ppm in a water base.

The compound of formula III: A strong sweet taste, approximately 200-300 times sweeter than sucrose was perceived when a samples of the purified compound were tasted at a concentration of 500 ppm in a water base.

The compound of formula VIII: A strong sweet taste, approximately 200-300 times sweeter than sucrose was perceived when a samples of the purified compound were tasted at a concentration of 500 ppm in a water base.

1. A method of enhancing a sweet taste of a flavored article, the method comprising introducing a glucosylated terpene glycoside to the flavored article, wherein the glucosylated terpene glycoside is: mono α-1,6 glucosylated stevioside, mono α-1,6 glucosylated rebaudioside A, mono α-1,6 glucosylated rebaudioside B, mono α-1,6 glucosylated rebaudioside C, mono α-1,6 glucosylated rebaudioside D, mono α-1,6 glucosylated rebaudioside E, mono α-1,6 glucosylated rebaudioside F, mono α-1,6 glucosylated rebaudioside M, mono α-1,6 glucosylated dulcoside A, mono α-1,6 glucosylated steviolbioside, mono α-1,6 glucosylated rubusoside, or any combination thereof.

2. The method of claim 1, wherein the glucosylated terpene glycoside is present in the flavored article at a concentration ranging from 1 ppm to 1000 ppm.

3. The method of claim 1, wherein the flavored article comprises a sweetener.
